(19) United States
(12) Patent Application Publication
(10) Pub. No.: US 2010/0145208 A1
(43) Pub. Date: Jun. 10, 2010

(54) Device For Predicting Tachyarrhythmias And/Or Atrial Arrhythmias

(76) Inventors: Alexander Schirdewan, Berlin (DE); Niels Wessel, Berlin (DE)

Correspondence Address: BUCHANAN INGERSOLL & ROONEY PC, P.O. BOX 1404, ALEXANDRIA, VA, 22313-1404, US

(21) Appl. No.: 11/667,740

(22) PCT Filed: Nov. 11, 2005
(86) PCT No.: PCT/DE2005/002040

§ 371 Date: Jun. 24, 2008

(30) Foreign Application Priority Data
Nov. 12, 2004 (DE) 10 2004 054 751.3

(51) Int. Cl. A61B 5/0402

(52) U.S. Cl. 600/516

(57) ABSTRACT

The invention relates to a device for predicting tachyarrhythmias and/or atrial arrhythmias with recording means that record electric cardiac signals of a heart and with evaluating means that evaluate the recorded electric cardiac signals. In order to transmit the recorded cardiac signals to the evaluating means, the recording means are connected to the evaluating means. The device also comprises time means (5) that furnish circadian time signals, whereby associated circadian time signals which are assigned to the recorded electric cardiac signals and/or other electric cardiac signals, which are derived from the recorded electric cardiac signals. The inventive device also comprises memory means (6), which are coupled to the evaluating means (4) and which contain normal circadian cardiac data and/or other normal circadian cardiac data, and comprises output means (7), which are coupled to the evaluating means (4) and which transmits electric reaction signals. These reaction signals are generated by the evaluating means (4) in response to a comparison of the circadian normal cardiac data containing the recorded electric cardiac signals and/or with the other circadian normal cardiac data containing the other electric cardiac signals while taking the associated circadian time signals into account, and these reaction signals are transmitted to the output means (7).

The invention relates to a device for predicting tachyarrhythmias and/or atrial arrhythmias as well as being an electric therapy object with such a device.

### BACKGROUND OF THE INVENTION

Such devices are usually designed as electrical therapy devices, in particular stimulators such as implantable cardiac pacemakers, cardioverter or defibrillators. A fundamental principle of these devices consists of recording electrical signals of the heart and sending electrical impulses to the heart depending on the recorded electrical signals.

First implantable cardioverter defibrillators (LCD) were designed approximately 40 years ago. The first implantation in an animal model took place in 1976 and in humans in 1980. The first programmable defibrillator was implanted in 1988-1991 for the first time with endocardiac electrodes. Since 1994 the strongly volume-reduced devices are implanted subpectorally. The system of the ICD consists of a generator and an appropriate system of electrodes. This system constantly supervises the electrical activity of the heart. When ventricular tachyarrhythmias occur the equipment can deliver the electrical therapy programmed for each case: a high-energy defibrillation, a low energy cardioversion or anti-tachycardiac stimulation. The equipment consists of hermetically sealed titanium box and a pour-in-head, in which the connection points for the electrode systems are located. Energy sources and reservoir capacitors are accommodated in the box, connected with electronics. The connection from the generator to the patient is made by means of technically different electrode systems. The electricogram signal is transmitted for evaluation to the generator. In this way, ventricular arrhythmias can be recognized and treated with the programmed therapy. The available capacity of the batteries usually permits one life span of several years, afterwards the device must be exchanged operationally by new equipment. Newer devices have an internal memory for recording patient data, therapy deliveries and therapy recordings, e.g. stored electrograms or beat-to-beat intervals.

A device for forecasting of tachyarrhythmias is described for example in the document EP 1 302 158 A2. This familiar device can be implemented as an implantable therapy instrument. It is usually instructed to record electrical signals of the heart and to send electrical impulses to the heart. An appropriate collection unit for the electrical signals is connectable with an electrode cable leading into a ventricle and/or an atrium of the heart. Such an electrode cable can be designed for the uni-, bi- or multi-polar recording of electrical signals from the heart and an appropriate delivery of therapeutic currents or impulses to the heart. To record electrical signals of the heart, bipolar electrode cables are suitable, for example, which can be switched together with the collection unit in such a way that uni- or bipolar recordings can be made of the captured signals.

For the delivery of stimulation or defibrillation impulses to a heart such therapy instruments usually includes a stimulation unit. The collection and stimulation units of such an implant are usually connected by a control unit, which manages the delivery of impulses to the heart dependent upon on the electrical signals from the heart and possibly additional signals. Such additional signals can be necessary in cases where a rate-adaptive cardiac pacemaker for the individual physiological needs of a patient is necessary. As the characteristic parameter, on which the delivery of stimulations or defibrillation impulses to the heart is executed, this device uses an increase of heart rate variability.

Moreover, a documented procedure and a device for evaluating the heart rate variability of the heart for the purposes of forecasting tachyarrhythmias is known from document of U.S. Pat. No. 6,144,878, there is. Again, this procedure and device uses deviations of a measured heart rate variability from a normally defined one to forecast tachyarrhythmias.

### SUMMARY OF THE INVENTION

The object of the invention is to provide a device for forecasting tachyarrhythmias and/or atrial arrhythmias with improved forecast accuracy and reliability.

The solution of this problem is achieved by a device for predicting tachyarrhythmias and/or atrial arrhythmias according to the features of claim 1. In accordance with a further aspect of the invention an electrical therapy instrument is provided, specifically a stimulator, implantable cardiac pacemaker, cardioverter or defibrillator. Preferred embodiments of the invention are subject to dependent sub-claims.

The invention consists of a device for predicting tachyarrhythmias and/or atrial arrhythmias by means of recording and data collection means of the electrical heart signals of a heart. The recorded electrical heart signals evaluation means are thus created, whereby the collection means are connected to the evaluation means for transferring the recorded heart signals to the evaluation means, and whereby the device exhibits the following additional features: Time means supplying circadian time signals, whereby associated circadian time signals are assigned to recorded electrical heart signals and/or to further electrical heart signals, which are derived from the recorded electrical heart signals. To the evaluation means coupled memory means, containing normal circadian heart data and/or further normal circadian heart data. Evaluation means coupled output means, delivering electrical reaction signals. The evaluation means produce the reaction signals in response to a comparison of the circadian normal heart data with the recorded electrical heart signals and/or the further normal circadian heart data with the additional electrical heart signals under consideration of the associated circadian time signals and transfer them to the output means.

The invention is based on the finding that the heart rhythm before the onset of life-threatening arrhythmias differs as a function depending on the occurrence of arrhythmias at different times of day. Based on this fact the device is designed in such a way that the time of day is involved in the forecast of tachyarrhythmias and/or atrial arrhythmias. Time-dependent control data are compared with current measured values for heart signals. The consideration of time-dependency in the forecasts enables a more accurate and individually patient-adapted prognosis of ventricular tachyarrhythmias and/or atrial arrhythmias.

As evaluated parameters for the forecasting of tachyarrhythmias and/or atrial arrhythmias preferably currently measured values and previously stored normal values for the heart rate (HR) and/or the heart rate variability (HRV) rhythms are considered. Here provision is made for the stored data for the normal values to be used as comparison with the currently measured values to be updated uniquely at regular intervals or in given time intervals, whereby an actualization can mean substitution of the previously stored normal data by updated normal data or the consideration of current normal data, e.g. previously stored and currently normal data are averaged.

Triggering mechanisms of tachyarrhythmias depend on the circadian rhythm. The measurement of the circadian heart rate and/or their variability serves the basis of a circadian individual profile and the diagnosis of the excess of individually specified standard values for the forecasting of tachyarrhythmias and a possible preventive differential therapy, in order to suppress a threatening ventricular arrhythmia.

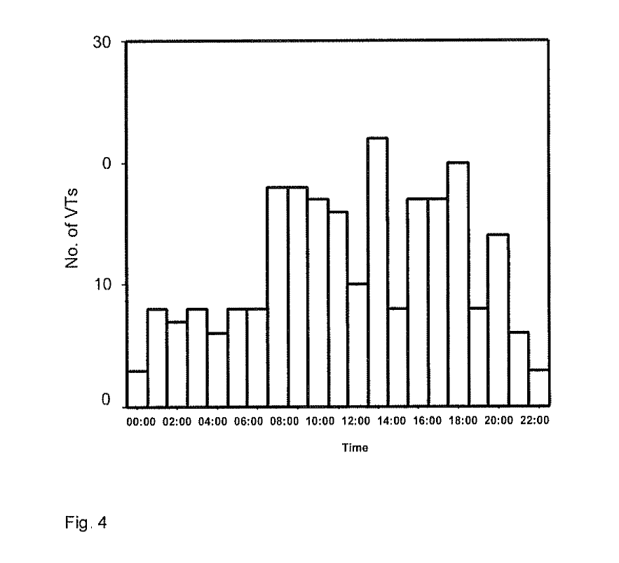

Furthermore atrial fibrillation (AF) is a significant rhythm disturbance, which is generated in the atria. The heart rate variability (HRV) is circadian dependent and changed before AF, whereby a forecast of AF can be predicted. The circadian distribution of the occurrence of AF reaches a maximum at night, i.e. a behaviour moving in opposite directions to ventricular arrhythmias (moving in opposite directions to FIG. 4 down).

The circadian HRV profile can be used both for the forecast of tachyarrhythmias and for AF. Such a profile can be realized by measurement of the heart rate and/or its variability from the R-peak (V-wave in the Pacer/ICD-chamber probe) or from the measurement of the P-waves (A-wave in the Pacer/ICD-atrial probe). The variability of the atrioventricular conduction (atrial-ventricular coupling, PR interval, AV-interval in the Pacer/ICD-atrial and chamber probe) gives additional circadian information together with HR/HRV, or on its own for the forecasting of AF and/or tachyarrhythmias.

Measurement of the circadian atrial frequency and/or its variability as well as the circadian atrial-ventricular coupling (PR-interval in the ECG, AV-interval in the pacemaker/ICD) and/or their variability can act as a basis for the production of a circadian individual profile for the forecasting of atrial arrhythmias (AF) and a possible preventive differential therapy to suppress a threatening atrial episode.

With the described device a procedure for the forecasting of tachyarrhythmias and/or atrial arrhythmias can be executed.

### DESCRIPTION OF PREFERRED DESIGN OPTIONS

Die Erfindung wird im folgenden anhand von Ausführungsbeispielen unter Bezugnahme auf die Figuren der Zeichnung näher erläutert, Hierbei zeigen:

The following gives a detailed description of the invention based on design examples with reference to the figures of the design drawing. The following is illustrated:

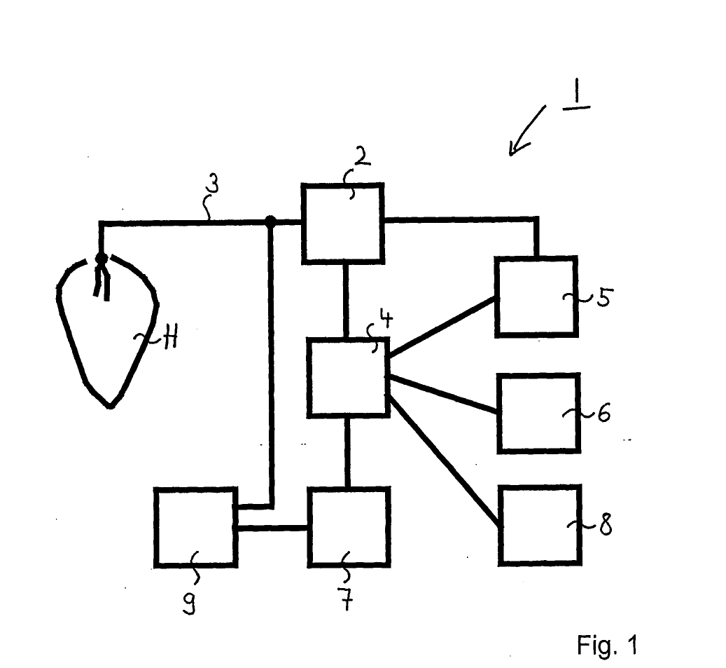

FIG. 1 A schematic block representation of a device for the forecast of tachy- and/or atrial arrhythmias;

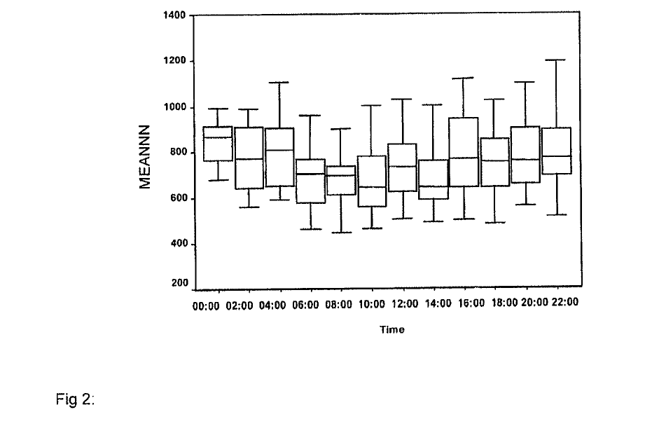

FIG. 2 A diagram of a circadian rhythm of an averaged heart rate before life-threatening arrhythmias;

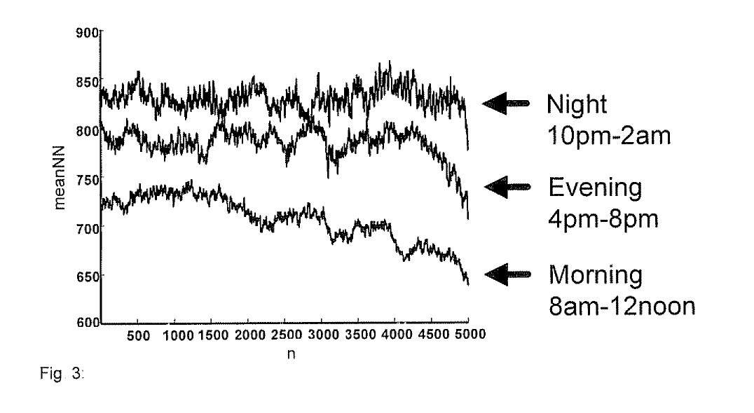

FIG. 3 A diagram of the averaged heart rate dynamics before life-threatening arrhythmias at different times of day;

FIG. 4 A diagram of the circadian distribution of the occurrence of life-threatening arrhythmias for different times of day and

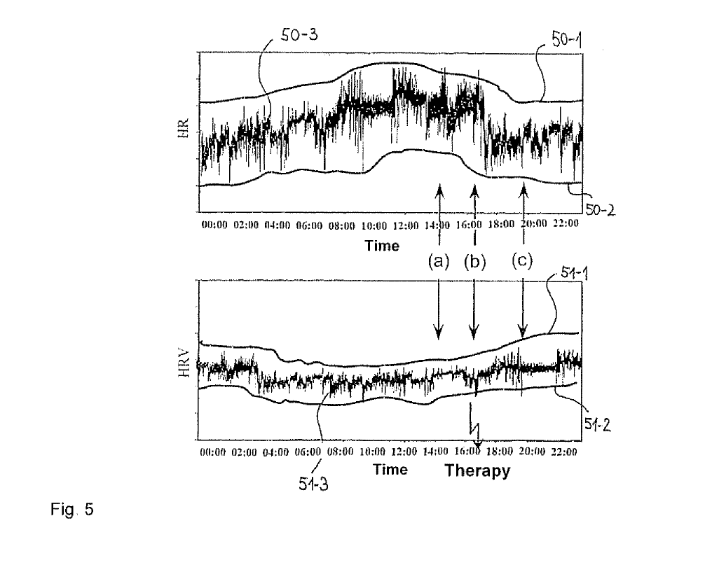

FIG. 5 A schematic representation of a circadian profile for the heart rate (HR) and the heart rate variability (HRV) at different times of day.

FIG. 1 shows a schematic representation for the explanation of a device 1 to predict tachyarrhythmias and/or atrial arrhythmias, with which it concerns for example an implantable cardioverter defibrillator (ICD). Collection means 2 for recording electrical signals of a heart H are connected with an electrode cable 3, which, for example, leads into the right ventricle of the heart. Collection means 2 have the usual means for taking up an intra-cardiac electrocardiogram as, for example, an input amplifier, as well as a QRS detector, in order to detect the QRS complex in intra-cardiac measured electrocardiogram.

With the help of the collection means 2 the current heart frequency/heart rate (HR) can be determined from the distance of a R-peak of the QRS complex to the R-peak of the next QRS complex. The determination of the heart rate can take place alternatively with the help of the collection unit 2 or in an evaluation unit 4. On the basis of the heart rate either with the help of the collection means 2 or with the evaluation means 4 the heart rate variability (HRV) can be determined, for example as the standard deviation of the measured heart rate.

The device 1 shown in FIG. 1 for the forecasting of tachyarrhythmias and/or atrial arrhythmias has in addition time means 5, which are connected with the collection means 2 and the evaluation means 4. By means of time means 5 circadian electrical time signals are produced, which are assigned to the recorded heart signals and/or the heart rate variability derived from this in the collection means 2 and/or the evaluation means 4 according to signals the respective collection time. In this way one gets pairs of variates, which cover the point of day time of the measurement recording and the associated measured value for the heart rate and/or the heart rate variability.

In the evaluation means 4 these produced pairs of measured values are compared with circadian control data for the heart rate and/or the heart rate variability, which are deposited in electronic memory means 6. Memory means 6 are connected for the transmission of the circadian normal/control data with evaluation means 4. If in the comparison of the current measured values with the circadian normal values an excess of given threshold values is determined, then, by means of evaluation means 4 an electrical reaction signal is produced and sent to output means 7 which are connected with evaluation means 4. Output means 7 are, in the simplest case, an exit for the delivery of electrical signals of evaluation means 4. In device 1 shown in FIG. 1, given threshold values are stored in threshold value means 8, which are coupled to evaluation means 4. Alternatively, memory means 6 and threshold value means 8 can be used as a common memory, as both the circadian normal data and threshold value data are stored.

The electrical reaction signal is given over to output means 7 at stimulation means 9, which are connected by the electrode cable 3. As a function of the electrical reaction signal stimulation means 9, an electrical stimulation signal, for example, is produced, in order to react to determined rhythm disturbances of the heart H.

FIG. 2 shows a diagram of a circadian rhythm of the averaged heart rate before life-threatening arrhythmias. The averaged beat-to-beat-interval, which is reciprocal to the heart rate, is demonstrated as a function of the daytime. In the diagram, a higher heart rate can be observed at day times. In this way, the heart rate dynamics follow the circadian rhythm. Furthermore, at night times no increase of the heart rate before life-threatening rhythm disturbances can be observed, while this increase happens ten minutes in the evening and one hour before in the morning (as visible from FIG. 3, which shows the heart rate dynamics (5000 values) before life-threatening arrhythmias in the morning, in the evening and at night. Again averaged beat-to-beat-intervals are presented.)

Surprisingly, it was stated that life-threatening arrhythmias have different precursors at different times of day. Beyond that there exists a circadian distribution of the occurrence of such arrhythmias, as shown in FIG. 4. In this figure the frequency of the occurrence of tachyarrhythmias as a function of time of day is represented.

FIG. 5 shows a schematic representation of a circadian profile for the heart rate (HR) and the heart rate variability (HRV) at different times of day. While the graphs 50-1 and 50-2 represent upper resp. lower normal/threshold value data for the heart rate, the graph 50-3 shows current recorded values of the heart rate. Similarly, the graph 51-1 and 51-2 form circadian normal/threshold values for the heart rate variability, whereas the graph 51-3 corresponds to currently measured values for the heart rate variability. At the time (a) the upper threshold value (graph 50-1) for the heart rate is exceeded, however, it evokes no electrical reaction signal, because the currently measured value for the heart rate variability (graph 51-3) falls in the standard range. In contrast to it at time (b) an electrical reaction signal with the evaluation means 4 (see FIG. 1) is evoked, because in addition to the currently measured value for the heart rate (graph 50-3) also the currently measured value for the heart rate variability (graph 51-3) falls below the lower threshold value (graph 51-2). In this execution example represented here for the time (c) no electrical reaction signal is evoked, because at this time the currently measured value for the heart rate (graph 50-3) lies in the standard range, whereas the currently measured value for the heart rate variability (graph 51-3) falls below the lower threshold value.

The advantages of considering the circadian dependence of the heart rhythm in the forecast of tachyarrhythmias are also evident, if with the help of device 1 in FIG. 1 an electronic reaction signal in the evaluation means 4 is produced, as soon as (in contrast to the design example represented in FIG. 5) either only the currently measured value of the heart rate (curve 50-3) or the currently measured value of the heart rate variability (curve 51-3 in FIG. 5) lies outside of the respective standard range. In both cases, however, the circadian dependence is taken into account.

The features of the invention disclosed in the forgoing description, claims and figures, can be used independently as well as in any combination for the purposes of implementing the different embodiments of the invention.

1. Device for predicting tachyarrhythmias and/or atrial arrhythmias marked by electrical heart signals of a heart recording collection means (1) and the recorded electrical heart signals evaluating evaluation means (4), whereby the collection means (1) are coupled to the evaluation means (4), characterized by
time means (5) supplying circadian time signals, whereby associated circadian time signals are assigned to the recorded electrical heart signals and/or further electrical heart signals, which are derived from the recorded electrical heart signals,—to the evaluation means (4) coupled memory means (6) with circadian normal heart data and/or further circadian normal heart data and
coupled to the evaluation means (4) and electrical reaction signals sent by output means (7), whereby the reaction signals of the evaluation means (4) are produced in reaction to a comparison of the circadian normal heart data with the captured electrical heart signals and/or the further circadian normal heart data with the further electrical heart signals with consideration of the associated circadian time signals and handed over to the output means (7).

2. Device according to claim 1, characterized by the fact, that the collection means (2) are instructed in order to capture electrical heart rate (HR)—signals as electrical heart signals.

3. Device according to claim 2, characterized by the fact, that the evaluation means (4) and/or the collection means (2) are instructed to derive electrical heart rate variability (HRV)—signals from the electrical heart rate (HR)—signals as the further electrical heart signals.

4. Device according to one of the preceding claims, characterized by the fact, that circadian-dependent normal heart rate (HR) data are stored as circadian normal heart data.

5. Device following one of the preceding claims, characterized by the fact, that circadian dependent normal heart rate variability (HRV)—data are stored as further circadian normal heart data.

6. Electrical therapy instrument, in particular stimulator, for example, implantable cardiac pacemaker, cardioverter or defibrillator, characterized by a device according to one of the preceding claims.
